(19) United States
(12) Patent Application Publication
(10) Pub. No.: US 2007/0077279 A1
(43) Pub. Date: Apr. 5, 2007

(54) Novel compositions containing polyphenols

(75) Inventors: Loni Schweikert, Zuzgen (CH); Peter Steinke, Grenzach-Wyhlen (DE)

Correspondence Address: NIXON & VANDERHYE, PC, 901 NORTH GLEBE ROAD, 11TH FLOOR, ARLINGTON, VA, 22203, US

(73) Assignee: DSM IP Assets B.V., TE Heerlen (NL)

(21) Appl. No.: 11/540,955

(22) Filed: Oct. 2, 2006

Related U.S. Application Data
(60) Provisional application No. 60/721,993, filed on Sep. 30, 2005.

(51) Int. Cl. A61K 36/87; A61K 36/82; A61K 9/20; A61K 9/68; A61K 36/63

(52) U.S. Cl. 424/440; 424/729; 424/766; 424/769; 424/464

(57) ABSTRACT

The present invention is directed to compositions containing at least a polyphenol and polyethylenglycol, to products such as food, beverages, dietary supplements, feed, pharmaceuticals and personal care products containing such a composition as well as to the use of polyethylenglycol for masking the bitter taste of such polyphenols. The polyphenols are preferably selected from the group consisting of epigallocatechin gallate, resveratrol, hydroxytyrosol, oleuropein, polyphenols present in green tea extracts, catechins, polyphenols present in extracts of red grape skin, polyphenols present in olives and/or olive waste water, and their mixtures.

### CROSS-REFERENCE TO RELATED APPLICATION

This application is related to and claims domestic priority benefits under 35 USC §119(e) from U.S. Provisional Application Ser. No. 60/721,993 filed on Sep. 30, 2005, the entire content of which is expressly incorporated hereinto by reference.

### FIELD OF THE INVENTION

The present invention is directed to compositions containing at least a polyphenol and polyethylenglycol, to products such as food, beverages, dietary supplements, feed, pharmaceuticals and personal care products containing such a composition as well as to the use of polyethylenglycol for masking the bitter taste of such polyphenols, especially in dry applications like powders, chewable tablets and lozenges.

### BACKGROUND AND SUMMARY OF THE INVENTION

Polyphenols often have a bitter taste which is not accepted by customers. The object of the present invention is therefore to provide a substance which can be added to a composition containing such a bitter tasting polyphenol whereby the bitter taste can be masked.

The object is solved by adding polyethylenglycol to a composition containing at least a polyphenol, especially at least a bitter tasting polyphenol. Preferably, the polyethylenglycol will be present in an amount to achieve a weight ratio of the polyphenol to the polyethylenglycol of from 10:90 to 70:30. The polyethylenglycol may advantageously be one of the following formula HO—(CH₂—CH₂—O—)ₙH with n being an integer, so that the average molecular weight of the polyethylenglycol is in the range from 1000 to 20000 g/mol.

In some embodiments of the invention, the polyphenol is selected from the group consisting of epigallocatechin gallate, resveratrol, hydroxytyrosol, oleuropein, polyphenols present in green tea extracts, catechins, polyphenols present in extracts of red grape skin, polyphenols present in olives and/or olive waste water, and their mixtures. The green tea extract that may be used satisfactorily in accordance with the invention may be one containing epigallocatechin gallate in an amount of at least 30 weight-%, preferably of at least 50 weight-%, based on the total amount of the green tea extract. The extract of red grape skin that may be used satisfactorily in accordance with the invention may be one containing resveratrol in an amount of at least 30 weight-%, preferably of at least 50 weight-%, based on the total amount of the extract of red grape skin.

The compositions of the invention may comprise vitamin C and/or vitamin E.

The compositions of the invention may be formulated into a product selected from the group consisting of dietary compositions, pharmaceuticals and personal care products. In some embodiments of the invention, the compositions of the invention may be in the form of a chewable tablet or a lozenge.

### DETAILED DESCRIPTION OF THE PREFERRED EXEMPLARY EMBODIMENTS

The polyphenols are preferably selected from the group consisting of polyphenols present in green tea extracts like catechins, polyphenols present in extracts of red grape skin like resveratrol, polyphenols present in olives, olive waste water etc. like hydroxytyrosol and oleuropein, and their mixtures.

Suitable green tea extracts are e.g. those containing epigallocatechin gallate (EGCG) in an amount of at least 30 weight-%, preferably in an amount in the range of from 30 weight-% to 100 weight-% (preferably in an amount in the range of from 35 weight-% to 60 weight-%), more preferably in an amount of at least 50 weight-%, most preferably in an amount in the range of from 50 weight-% to 99 weight-%, based on the total amount of the green tea extract. Those green tea extracts may also contain caffeine in an amount up to 15 weight-%, preferably in an amount in the range of from 0.1 to 12 weight-%, more preferably in an amount of from 0.1 to 3 weight-%, based on the total weight of the green tea extract. The total amount of tea polyphenols in such green tea extracts may be preferable in the range of from 85 to 98 weight-% (preferably in the range of from 90 to 98 weight-%), whereas the total amount of catechins may be preferably in the range of from 65 to 90 weight-% (preferably in the range of from 65 to 80 weight-%), based on the total weight of the green tea extract.

The term “(−)-epigallocatechin gallate” (EGCG) encompasses also (−)-EGCG derivatives such as pharmaceutically acceptable salts.

Catechins are especially found in green tea extracts such as epicatechin gallate (ECG), epigallocatechin (EGC), gallocatechin gallate (ECG) and epigallocatechin gallate (EGCG), whereby EGCG is the most preferred one. EGCG can also be used in a formulated product form such as a Teavigo® tablet grade (=a green tea extract containing ca. 88% of EGCG admixed with ca. 3% of pectin), which is a directly compressible form granulated with pectin and is commercially available from DSM Nutritional Products Ltd, Kaiseraugst, Switzerland.

Another suitable (−)-EGCG is e.g. Teavigo (a green tea extract containing ≧94% of EGCG), commercially available from DSM Nutritional Products Ltd, Kaiseraugst, Switzerland.

Another preferred embodiment for (−)-epigallocatechin gallate is a green tea fraction comprising at least 90 weight-% of (−)-epigallocatechin gallate (EGCG) and at most 2 weight-% of caffeine, especially a green tea fraction comprising at least 90 weight-% of EGCG, at most 1 weight-% of caffeine and at most 10 weight-% of epicatechin gallate (ECG), more especially a green tea fraction comprising at least 90 weight-% of EGCG, at most 0.5 weight-% (preferably at most 0.1 weight-%) of caffeine, at most 5.0 weight-% of epicatechin gallate (ECG) (preferably in the range of from 0.1 to 2.5 weight-%) and at most 3.5 weight-% (preferably at most 1.0 weight-%) of a total amount of epicatechin (EC), catechin (CAT), catechin gallate (CG), epigallocatechin (EGC), gallocatechin gallate (GCG), gallocatechin (GC) and gallic acid (GA) together, based on the total weight of the green tea extract.

Resveratrol can be used in its essentially pure form derived from natural sources or from chemical synthesis, in a product form containing resveratrol and further additives, e.g. as a directly compressible form or as an extract.

The extracts of red grape skin are especially those containing resveratrol in an amount of at least 30 weight-%, preferably in an amount in the range of from 30 weight-% to 100 weight-%, more preferably in an amount of at least 50 weight-%, most preferably in an amount in the range of from 50 weight-% to 99 weight-%, based on the total amount of the red wine extract.

The term “resveratrol” as used herein comprises a derivative, metabolite or analogue thereof. The carbon-carbon double bond may be trans or cis and includes cis/trans mixtures. Etherified or esterified hydroxy groups may be derived from non-substituted or substituted, straight or branched chain alkyl groups having 1 to 26 carbon atoms or from non-substituted or substituted, straight or branched chain aliphatic, araliphatic or aromatic carboxylic acids having 1 to 26 carbon atoms. Etherified hydroxy groups may further be glycoside groups and esterified hydroxy groups may further be glucuronide or sulfate groups. Of primary interest for the purposes of the invention is (trans)-resveratrol.

Hydroxytyrosol and/or oleuropein can be used in its/their essentially pure form derived from natural sources or from chemical synthesis, in a product form containing it/them and further additives, e.g. as a directly compressible form or as an extract, i.e. hydroxytyrosol may be of synthetic origin or it may be obtained together with other water-soluble polyphenols such as tyrosol and oleuropein from extraction of olive leaves, olive fruits and vegetation water of olive oil production.

In embodiments of the present invention mixtures of hydroxytyrosol with oleuropein, preferably in a weight ratio in the range of from 1:1 to 200:1, more preferably in a weight ratio in the range of from 5:1 to 200:1, most preferably in a weight ratio in the range of from 10:1 to 100:1, may be used.

In other embodiments of the present invention mixtures of hydroxytyrosol with tyrosol, preferably in a weight ratio in the range of from 1:1 to 50:1, more preferably in a weight ratio in the range of from 3:1 to 50:1, most preferably in a weight ratio in the range of from 5:1 to 30:1, may be used.

Examples of references that deal with the extraction of oleuropein and/or hydroxytyrosol from olive leaves are WO 02/18310, US 2002/0198415, WO 2004/005228, U.S. Pat. No. 6,416,808 and US 2002/0058078 which disclose a method for acidic hydrolysis of olive vegetation water for 2 to 12 months until at least 90% of the present oleuropein has been converted. A method of extraction of phenolic compounds from olives, olive pulps, olive oil and oil mill waste water is described by Usana Inc. patents U.S. Pat. No. 6,361,803 and WO 01/45514 and in US 2002/0004077. EP-A 1 582 512 describes an extraction of hydroxytyrosol from olive leaves. A method for obtaining hydroxytyrosol and/or oleuropein from the vegetation water of de-pitted olives is disclosed in US 2004/0039066 A1 in paragraphs [0080]-[0091].

Derivatives of hydroxytyrosol may be esters as well as physiologically/pharmaceutically acceptable salts. Preferred examples are the mono-, di- and triesters of hydroxytyrosol with (un)saturated carbonic acids R—COOH, whereby R is an alkyl or alkenyl chain having 2 to 22 carbon atoms.

Commercially available hydroxytyrosol containing olive extracts which may be used according to the invention include e.g. extracts from olive fruits such as Polyphen-Oil™ from Life Extension, OleaSelect™ from Indena, Hytolive® from Genosa, Prolivols from Seppic, OLIVE LEAF or OLIVE Water Extract of Olea europea from Lalilab, Iitofulvic from Ebiser, hydrolysed olive leaf extract, such as described in EP1582512, olive leaf extract, rich in oleuropein, such as available from Furfural and HIDROX® from CreAgri. Preferably HIDROX® commercially available from CreAgri such as HIDROX® 2% spray dried powder, HIDROX® Gold freeze dried powder (9%) and HIDROX® 6% freeze dried powder organic olive juice extract are used.

HIDROX® 2% spray dried powder organic olive juice extract (product) is a concentrate of the waste water obtained in the olive oil production containing dry solids in the range of from 30 to 35 weight-% (preferably in the range of from 32 to 33 weight-%), whereby at least 20 to 30 weight-% of these dry solids are polyphenols so that the total amount of the polyphenols is around 6 weight-% in the product, maltodextrin in the range of from 60 to 70 weight-% (preferably in the range of from 63 to 69 weight-%) and citric acid in the range of from 0.5 to 2.5 weight-% (preferably in the range of from 1 to 2 weight-%), based on the total weight of the product.

HIDROX® Gold freeze dried powder (9%) organic olive juice extract (product) is a concentrate of the waste water obtained in the olive oil production containing dry solids in the range of from 97.5 to 99.5 weight-% (preferably in the range of from 98 to 99 weight-%), whereby at least 7 to 15 weight-% (preferably 10 to 12 weight-%) of these dry solids are polyphenols so that the total amount of the polyphenols is around 9 weight-% in the product, and citric acid in the range of from 0.5 to 2.5 weight-% (preferably in the range of from 1 to 2 weight-%), based on the total weight of the product.

HIDROX® 6% freeze dried powder organic olive juice extract (product) is a concentrate of the waste water obtained in the olive oil production containing dry solids in the range of from 97.5 to 99.5 weight-% (preferably in the range of from 98 to 99 weight-%), whereby at least 15 to 20 weight-% (preferably 15.5 to 17 weight-%) of these dry solids are polyphenols so that the total amount of the polyphenols is around 6 weight-% in the product, and citric acid in the range of from 0.5 to 2.5 weight-% (preferably in the range of from 1 to 2 weight-%), based on the total weight of the product.

The types of polyethylenglycol are especially of the following formula HO—(CH₂—CH₂—O—)ₙH with an average molecular weight from 1000 to 20000. The most preferred polyethylenglycol is PEG 6000, e.g. commercially available from Clariant GmbH, 65840 Sulzbach, Germany. The number X in the type name “PEG X” indicates the average molecular weight of the polymer.

The weight ratio of the polyphenol to the polyethylenglycol may be from 10:90 to 70:30, preferably from 20:80 to 60:40, most preferably from 25:75 to 60.40.

In preferred embodiments of the present invention the composition further comprises vitamin C and/or vitamin E.

The expression “vitamin C” encompasses (L-)ascorbic acid as well as their salts and esters like ascorbyl palmitate and stearate as well as any further derivatives and product forms thereof, like directly compressible powders.

The expression “vitamin E” encompasses all-rac-tocopherol and tocopherols derived from natural sources as well as their esters like acetates and succinates as well as any further derivatives and product forms thereof, like directly compressible and/or water-dispersible powders.

In compositions containing at least a polyphenol, polyethyleneglycol, vitamin E and/or vitamin C the concentrations of the active ingredients per serving (e.g. as a chewable tablet) may vary from

1 mg to 300 mg for the polyphenol, e.g. EGCG,

10 mg to 360 mg for vitamin C,

1 mg to 100 mg for vitamin E (calculated as mg Tocopherol equivalent).

Preferably the concentrations of the active ingredients per serving (e.g. as a chewable tablet) may vary from

1 mg to 150 mg for the polyphenol, e.g. EGCG,

10 mg to 240 mg for vitamin C,

1 mg to 50 mg for vitamin E (calculated as mg Tocopherol equivalent).

Most preferably the concentrations of the active ingredients per serving (e.g. as a chewable tablet) may vary from

1 mg to 80 mg for the polyphenol, e.g. EGCG,

10 mg to 180 mg for vitamin C,

10 mg to 30 mg for vitamin E (calculated as mg Tocopherol equivalent).

That means that the weight ratio of vitamin E to vitamin C may vary from (1:1) to (1:10), preferably from (1:3.6) to (1:10),more preferably from (1:4.8) to (1:6), and/or that the weight ratio of vitamin E to the polyphenol (preferably being EGCG) may vary from (10:1) to (1:3), preferably from (1:1) to (1:3), more preferably from (1:1) to (1:2.6).

The compositions of the present invention may be prepared by a process comprising the following steps:
a) providing the polyphenol and optionally mixing it with vitamin C and/or vitamin E;
b) optionally adding a sweetener, a flavour and/or other excipients and mixing;
c) adding the polyethylenglycol and mixing the thus obtained mixture.

The compositions of the present invention may also be prepared by mixing processes known to the person skilled in the art for the corresponding type of application and may be conducted for powder mixtures according to the procedures mentioned in the given examples.

The composition of the present invention may be pressed to tablets. Therefore, the present invention is also directed to a process for the manufacture of tablets comprising the following steps:
a) providing the polyphenol and optionally mixing it with vitamin C and/or vitamin E;
b) optionally adding a sweetener, a flavour and/or other excipients and mixing;
c) adding the polyethylenglycol and mixing the thus obtained mixture;
d) optionally sieving the mixture obtained in step c);
e) compressing the sieved mixture to tablets.

Surprisingly it was discovered that in compositions such as lozenges and chewable tablets containing low amounts of polyethylenglycol the bitter taste can be masked even better if polyethylenglycol is added at the beginning of the formulation process than in the end of the formulation process. This may be done by premixing the bitter polyphenols like EGCG with PEG for a few minutes, for example in a tumbler mixer prior to the further processing steps.

The present invention is also directed to products selected from the group consisting of dietary compositions, pharmaceuticals and personal care products containing such a composition as defined above. Preferred are products such as chewable tablets or lozenges.

The term “dietary compositions” comprises any type of (fortified) food, (fortified) (animal) feed and beverages including also clinical nutrition, and also dietary supplements as well as the corresponding additives: food additives, beverage additives, feed additives. Also encompassed is functional food/feed i.e. a food/feed that has been enhanced with vitamins, other micronutrients or pharmaceuticals to provide further specific health benefits, as well as a nutraceutical, i.e. a pill or other pharmaceutical product that has nutritional value.

The dietary compositions according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, weighting agents, jellyfying agents, gel forming agents, antioxidants and antimicrobials.

A further object of the present invention is the use of polyethylenglycol for masking the bitter taste of polyphenols. Specifically, according to an embodiment of the invention, the bitter taste of polyphenols may be masked by adding a taste-masking effective amount of polyethylenglycol to a composition which comprises the bitter tasting polyphenol. Preferably, the polyethylenglycol will be present in a taste-masking effective amount to achieve a weight ratio of the polyphenol to the polyethylenglycol of from 10:90 to 70:30.

### EXAMPLES

### Example 1

### Preparation of Lozenges Containing EGCG, Vitamin E and Vitamin C

| | | Quantities |
|---|---|---|
| | Composition | [mg/Tablet] |
| A | EGCG as TEAVIGO TG | 12.50 |
| B | Vitamin E (Tocopherol Acetate) as Vitamin E 50% | 3.30 |
| | CWS/F | |
| C | Vitamin C as Ascorbic Acid fine granular | 10.50 |
| D | Sorbitol DC as Neosorb 60 W (1) | 168.80 |
| E | Siliconodioxide as Aerosil 200 (2) | 1.10 |
| F | Aroma as Frescoforte Aroma Permaseal 60470-31 (3) | 10.00 |
| G | Aroma as Eiszucker Aroma Permaseal 60153-73 (4) | 6.00 |
| H | Sweetener as Twinsweet (5) | 1.60 |
| I | PEG 6000 (6) | 20.00 |
| J | Mg-Stearate (7) | 1.20 |
| | Total Tablet Weight | 235.00 |

(1) Neosorb 60W: Roquette Frères, 4 Rue Patou, F-59022 Lille Céded, France;
(2) Aerosil 200, Degussa AG, 40402 Duesseldorf, Germany;
(3) Frescoforte Aroma Permaseal: Givaudan Schweiz AG, Ueberlandstrasse 138, CH-8600 Duebendorf, Switzerland;
(4) Eiszucker Aroma Permaseal: Givaudan Schweiz AG, Ueberlandstrasse 138, CH-8600 Duebendorf, Switzerland;
(5) Twinsweet: Holland Sweetener Company, P.O. Box 258, 6160 Geleen, Nederlands;
(6) PEG 6000: Clariant GmbH, 65840 Sulzbach, Germany;
(7) Magnesium Stearate: Tracomme AG, CH-8134 Adliswil.

Procedure

I Pos. A-C were weighed into a drum and mixed for 2 minutes

II Pos. D+E were sieved through a sieve with diameters of 0.80 mm in a second drum, and mixed for 5 minutes.

III Pos. F-I to I were added and mixed for 10 minutes.

IV II and III were put together and mixed for 10 minutes.

V Pos. J was sieved through a sieve with diameters of 0.80 mm, added to IV and mixed for 5 minutes.

VI The resulting mixture was then compressed to tablets under the conditions given below.

Tabletting Characteristics

Tabletting machine: COMPREX I

Punch: 8 mm R 9.5 round

Compression force: 10 KN

Hardness: 193.5 N

Thickness: 4.22 mm

Friability: 0.09%

Disintegration: 5′31″

### Example 2

### Preparation of Chewable Tablets Containing EGCG, Vitamin E and Vitamin C

| | | Quantities |
|---|---|---|
| | Composition | [mg/Tablet] |
| 1 | EGCG as TeavigoTM TG | 25.00 |
| 2 | Vitamin C as Ascorbic Acid Fine Granular | 33.00 |
| 3 | Vitamin E as Dry Vitamin E 50% SD | 15.65 |
| 4 | Polyethylenglykol as PEG 6000 (1) | 25.00 |
| 5 | Microcrystalline Cellulose as Avicel PH 200 (2) | 81.35 |
| 6 | Sweetener as Twinsweet (3) | 4.00 |
| 7 | Aroma as Grapefruit Flavour Prem. 76629-71 | 4.00 |
| | (Givaudan) (4) | |
| 8 | Aroma as Icesugar Flavour 60153-71 (Givaudan) (4) | 8.00 |
| 9 | Citric Acid as Citric Acid Fine Granular N51 (5) | 7.50 |
| 10 | Sorbitol as Neosorb P60W (6) | 542.70 |
| 11 | Mg Stearat (7) | 3.80 |
| | Total Tablet Weight | 750.00 |

(1) PEG 6000: Clariant GmbH, D-65840 Sulzbach, Germany;
(2) Avicel PH 102: FMC Europe NV, Avenue Louise 480 B9, B-1050 Brussels, Belgium;
(3) Twinsweet: Holland Sweetener Company, P.O. Box 258, 6160 Geleen, Nederlands;
(4) Aroma: Givaudan Schweiz AG, Ueberlandstrasse 138, CH-8600 Duebendorf, Switzerland;
(5) Citric Acid: Citrique Belge N.V., B-3300 Tienen;
(6) Neosorb P90W: Roquette Frères, 4 Rue Patou, F-59022 Lille Cédex, France;
(7) Magnesium Stearate: Tracomme AG, CH-8134 Adliswil.

Mixing Procedure

1-5 were weighed into a suitable vessel and mixed for 10 minutes.

6-9 were passed through a sieve with diameters of 1.0 mm and mixed 10 minutes in a separate vessel.

I and II were combined and mixed again for 10 minutes.

10 was passed through a sieve with diameters of 1.0 mm, then added to III and mixed for 10 minutes.

11 was passed through a sieve with diameters of 0.63 mm, added to IV and mixed for additional 5 minutes.

Afterwards the thus obtained mixture was compressed to tablets under the conditions given below.

Tabletting Characteristics

Tabletting machine: COMPREX I

Punch: 14 mm beveled edge

Compression force: 13.0 KN

Hardness: 218 N

Friability: 0.13%

### Example 3

### Stability Data of the Lozenges Prepared According to Example 1

Storage tests with lozenges produced according to example 1 were conducted under room temperature (25° C./60% relative humidity) in closed polypropylene tubes and showed good stability.

TABLE 1
25° C./60% (relative humidity) (0 to 6 months)

| Vitamin/ | | | | | |
|---|---|---|---|---|---|
| polyphenol in | Initial | | | | |
| mg/tablet expect | Assay | 1 month | 2 months | 3 months | 6 months |
| otherwise stated | (mg) | (mg) | (mg) | (mg) | (mg) |
| EGCG | 11.9 | 11.8 | 10.9 | 11.0 | 10.6 |
| Vit E | 1.6 | 1.7 | 1.7 | 1.8 | 1.6 |
| Vit C | 12.2 | 11.4 | 12.2 | 11.3 | 12.3 |

TABLE 2
25° C./60% relative humidity (0 to 24 months)

| Vitamin/polyphenol in | | | | |
|---|---|---|---|---|
| mg/tablet expect | Initial Assay | 12 months | 18 months | 24 months |
| otherwise stated | (mg) | (mg) | (mg) | (mg) |
| EGCG | 11.9 | 11.6 | 12.0 | 11.3 |
| Vit E | 1.6 | 1.5 | 1.6 | 1.7 |
| Vit C | 12.2 | 12.1 | 12.0 | 11.9 |

### Example 4

### Stability Data of the Chewable Tablets Prepared According to Example 2

Storage tests with chewables produced according to example 2 were conducted under room temperature (25° C./60% relative humidity) in closed polypropylene tubes and showed good stability.

TABLE 3
25° C./60% relative humidity

| Vitamin/ | | | | |
|---|---|---|---|---|
| polyphenol in mg/tablet | Initial Assay | 1 month | 2 months | 3 months |
| expect otherwise stated | (mg) | (mg) | (mg) | (mg) |
| EGCG | 23.8 | 23.0 | 23.8 | 24.1 |
| Vitamin C | 33.7 | 32.3 | 33.3 | 36.1 |
| Tocopherol-Acetate | 8.3 | 8.4 | 8.5 | 8.6 |

TABLE 4
25° C./60% relative humidity

| Vitamin/polyphenol in mg/tablet | Initial Assay | 6 months | 12 months |
|---|---|---|---|
| expect otherwise stated | (mg) | (mg) | (mg) |
| EGCG | 23.8 | 23.7 | 22.6 |
| Vitamin C | 33.7 | 32.5 | 33.0 |
| Tocopherol-Acetate | 8.3 | 8.3 | 8.6 |

### Example 5

### Stability Data of the Lozenges Prepared According to Example 1

Storage tests with lozenges produced according to example 1 were conducted under accelerated conditions (40° C./75% relative humidity) in closed polypropylene tubes and showed good stability.

TABLE 5
40° C./75% relative humidity

| Vitamin/polyphenol | | 1 month | 2 months | 3 months |
|---|---|---|---|---|
| in mg/tablet | Initial | (mg) | (mg) | (mg) |
| except otherwise stated | Assay (mg) | 40° C. | 40° C. | 40° C. |
| EGCG | 11.9 | 11.8 | 11.4 | 10.6 |
| Vit E | 1.6 | 1.6 | 1.6 | 1.8 |
| Vit C | 12.2 | 11.2 | 11.1 | 11.2 |

### Example 6

### Stability Data of the Chewable Tablets Prepared According to Example 2

Storage tests with chewables produced according to example 2 were conducted under accelerated conditions (40° C./75% relative humidity) in closed polypropylene tubes and showed good stability.

TABLE 6
40° C./75% relative humidity

| Vitamin/polyphenol | | 1 month | 2 months | 3 months |
|---|---|---|---|---|
| in mg/tablet except | Initial Assay | (mg) | (mg) | (mg) |
| otherwise stated | (mg) | 40° C. | 40° C. | 40° C. |
| EGCG | 23.8 | 24.8 | 23.2 | 23.2 |
| Vitamin C | 33.7 | 31.8 | 32.4 | 33.6 |
| Tocopherol-Acetate | 8.3 | 8.7 | 8.5 | 8.5 |

1. A composition comprising a polyphenol and polyethylenglycol.

2. The composition according to claim 1, wherein the polyphenol is selected from the group consisting of resveratrol, hydroxytyrosol, oleuropein, polyphenols present in green tea extracts, catechins, polyphenols present in extracts of red grape skin, polyphenols present in olives and/or olive waste water, and their mixtures.

3. The composition according to claim 2, wherein the green tea extract is one containing epigallocatechin gallate in an amount of at least 30 weight-%, preferably of at least 50 weight-%, based on the total amount of the green tea extract.

4. The composition according to claim 2, wherein the extract of red grape skin is one containing resveratrol in an amount of at least 30 weight-%, preferably of at least 50 weight-%, based on the total amount of the extract of red grape skin.

5. The composition according to claim 1 further comprising vitamin C and/or vitamin E.

6. The composition according to claim 1 wherein the weight ratio of the polyphenol to the polyethylenglycol is from 10:90 to 70:30.

7. The composition according to claim 1 wherein the polyethylenglycol is one of the following formula HO—(CH₂—CH₂—O—)ₙH with an average molecular weight of from 1000 to 20000.

8. A product selected from the group consisting of dietary compositions, pharmaceuticals and personal care products, the product comprising a composition according to claim 1.

9. Product according to claim 8 in the form of a chewable tablet or a lozenge.

10. A method for masking the bitter taste of polyphenol which comprises adding a taste-masking effective amount of polyethylenglycol to a composition which comprises a bitter tasting polyphenol.

11. Method according to claim 10, wherein the weight ratio of the polyphenol to the polyethylenglycol is from 10:90 to 70:30.
